(19) United States
(12) Patent Application Publication
(10) Pub. No.: US 2002/0009706 A1
(43) Pub. Date: Jan. 24, 2002

(54) Method for loading cells with an agent

(76) Inventors: Anthony P. McHale, Portstewart (IE); Roger K. Craig, Cheshire (GB); Emma Fadlon, Portstewart (IE)

Correspondence Address: PALMER & DODGE, LLP, ONE BEACON STREET, BOSTON, MA, 02108-3190, US

(21) Appl. No.: 09/779,188

(22) Filed: Feb. 8, 2001

Related U.S. Application Data
(60) Provisional application No. 60/181,796, filed on Feb. 11, 2000.

(30) Foreign Application Priority Data
Feb. 8, 2000 (GB) 9917416.1

(51) Int. Cl. A01N 1/02; C12N 1/00

(52) U.S. Cl. 435/2; 435/810

(57) ABSTRACT

A method is provided for selectively releasing an agent from a red blood cell comprising the steps of:

(a) presensitising a red blood cell by electrosensitising the cell;

(b) loading the cell with an agent;

(c) electrosensitising the cell; and

(d) causing the agent to be released from the sensitised cell by applying ultrasound at a frequency and energy sufficient to cause disruption of the sensitised cell but insufficient to cause disruption of unsensitised red blood cells, wherein steps (b) and (c) can be performed in any order.

### RELATED APPLICATIONS

This application claims priority under 35 U.S.C. § 120 to U.S. patent application Ser. No. of 09/748,063, and U.S. patent application Ser. No. 09/748,789, both filed Dec. 22, 2000, and under 35 U.S.C. § 119(e) to U.S. Provisional Application 60/181,796, filed Feb. 11, 2000. This application also claims priority under 35 U.S.C. § 119(a)-(d) to UK 9917416.1, filed Feb. 8, 2000. The entireties of these applications are incorporated by reference herein.

### FIELD OF THE INVENTION

The present invention relates to a method for delivering an agent in a red blood cell loaded with the agent, which cell is sensitised to assist in agent release.

### BACKGROUND TO THE INVENTION

The delivery of therapeutic agents to specific tissues is desirable typically to ensure that a sufficiently high dose of a given agent is delivered to a selected tissue. Moreover, it is often the case that the therapeutic agent, although advantageously having beneficial therapeutic effects on the diseased tissue, may have undesirable side effects on tissues that are not diseased. For example, in the treatment of certain types of disorders, such as cancer, it is necessary to use a high enough dose of a drug to kill the cancer cells without killing an unacceptable high number of normal cells. Thus, one of the major challenges of disease treatment is to identify ways of exploiting cellular drug delivery vehicles to incorporate and to selectively release agents at a desired target site.

It has been suggested that red blood cells may be exploited as active agent/drug delivery vehicles (DeLoach & Sprandel 1985, Bibliotheca Haematologica; Publ. Karger, Munich) as it is possible to incorporate agents into human red blood cells using a variety of techniques. An example of such a technique is the exploitation of osmotic shock and modifications thereof such as hypotonic shock and subsequent recovery of isotonicity and reverse hypotonic dialysis (Luque & Pinilla, 1993, Ind. Farmac. 8, 53-59).

An alternative method for loading drugs and active agents into red blood cells is electroporation. Using this process, the agent of interest are mixed with the live red blood cells in a buffer medium and short pulses of high electric fields are applied. The red blood cell membranes are transiently made porous and the agents of interest enter the cells. The electroporation process is advantageous as very high loading indices can be achieved within a very short time period (Flynn et al., 1994, Cancer Letts., 82, 225-229).

When packaging/carrier/delivery systems such as red blood cells are used as in vivo delivery systems, they suffer from the drawback that the delivery function is dependent upon both an accumulation of the red blood cells and a breakdown of the red blood cell membrane in or at the relevant tissue/site. As a result, attempts have been made to incorporate sensitising agents into cell carriers in order to facilitate both the accumulation and/or release of an agent of interest at a target site.

By way of example, our co-pending UK Patent Applications 9816583.0 and 9826676.0 relate inter alia to the incorporation of a dye compound, such as a porphyrin, which renders a loaded red blood cell susceptible to laser light treatment at a target site. This phenomenon, known as photodynamic activation, is exploited in order to achieve accumulation of the carrier vehicle at the relevant site and to achieve load release at that site.

Alternative energy sources have been investigated as tools for inducing payload release from loaded and sensitised cells. By way of example, ultrasound irradiation has been investigated as an alternative to light induced photodynamic activation as it has a broader degree of focus and it penetrates more deeply into the body. However, although ultrasound irradiation has also been applied to effect red blood cell lysis in vitro, its use has been limited in that its effect is only significant at lower cell concentrations (1-6×10⁶ cells) (Brayman et al., 1996, Ultrasound in Med & Biol., 22: 497-514). Moreover, ultrasound is non-specific in effects, resulting in lysis of both loaded and endogenous red blood cells.

Recently, it has been found that certain dye compounds, in particular porphyrins, can achieve a cytopathogenic effect when the disease site is subjected to ultrasound irradiation. This technique is referred to as sonodynamic therapy and is discussed in WO98/52609. WO98/52609 teaches that ultrasound irradiation may be useful in treating disease but only when it is combined with an effective amount of an ultrasound-susceptibility modification agent such as a porphyrin.

In our co-pending application, U.K. patent application No. 9917416.1, we have shown that treatment of red blood cells with an electric field increases their sensitivity to ultrasound mediated disruption. Consequently, efficient unloading of therapeutic agents carried by red blood cells at a site of interest can be achieved at lower exposures of ultrasound, reducing possible damage to normal red blood cells.

### SUMMARY OF THE INVENTION

We have now further shown that if the red blood cells are presensitised prior to a dialysis loading step, close to 100% efficiency in the loading step can be achieved. However we have also found that the dialysis loading step reduces the sensitivity of the loaded cells to ultrasound. This reduction in sensitivity can be reversed by subjecting the cells to an additional sensitisation step regardless of whether the additional step is performed before or after loading. Using this double sensitisation procedure, red blood cells can be produced that have both excellent loading characteristics and ultrasound sensitivity. Consequently, highly efficient unloading of therapeutic agents carried by red blood cells at a site of interest can be achieved at low exposures of ultrasound. This represents a considerable improvement over prior art methods. The present invention therefore provides an improved method for selectively releasing an agent from a loaded red blood cell at a target site.

Accordingly, the present invention provides in a first aspect a method for selectively releasing an agent from a red blood cell comprising the steps of:

(a) presensitising a red blood cell by electrosensitising the cell;

(b) loading the cell with an agent;

(c) electrosensitising the cell; and

(d) causing the agent to be released from the sensitised cell by applying ultrasound at a frequency and energy sufficient to cause disruption of the sensitised cell but insufficient to cause disruption of unsensitised red blood cells, wherein steps (b) and (c) can be performed in any order.

The present invention also provides in a second aspect a method for delivering an agent to a target site in a vertebrate, comprising the steps of:

(a) presensitising a red blood cell by electrosensitising the cell;

(b) loading the cell with an agent;

(c) electrosensitising the cell;

(d) introducing the cell into a vertebrate; and

(e) causing the agent to be released from the sensitised cell by applying ultrasound at a frequency and energy sufficient to cause disruption of the sensitised cell but insufficient to cause disruption of unsensitised red blood cells, wherein steps (b) and (c) can be performed in any order.

In a preferred embodiment, the red blood cell delivery vector is PEGylated prior to being introduced into the vertebrate.

Preferably the electrosensitisation procedures in step (a) and (c) of the above aspects are in vitro or ex-vivo procedures.

Preferably the electrosensitisation comprises the step of applying an electric field to the red blood cell, more preferably the electric field strength is from about 0.1 kV/cm to about 10 kV/cm under in vitro conditions. Preferably the electric field is applied for between 1 μs and 100 ms.

Preferably the ultrasound is selected from diagnostic ultrasound, therapeutic ultrasound and a combination thereof.

Preferably the applied ultrasound energy source is at a power level of from about 0.05 W/cm² to about 100 W/cm².

In a preferred embodiment the sensitisation of the red blood cell in step (c) of the above methods is performed after the loading of the agent in step (b).

In an alternative preferred embodiment the loading of the agent in step (b) of the above methods is performed after the sensitisation of the red blood cell in step (c).

In a further aspect, the present invention provides a method for preparing a red blood cell delivery vector which method comprises

(a) presensitising a red blood cell by electrosensitising the cell;

(b) loading the cell with an agent; and

(c) electrosensitising the cell,

wherein steps (b) and (c) can be performed in any order.

The present invention also provides a red blood cell delivery vector obtainable by a method comprising

(a) presensitising a red blood cell by electrosensitising the cell;

(b) loading the cell with an agent; and

(c) electrosensitising the cell,

wherein steps (b) and (c) can be performed in any order.

Also provided is a kit comprising a red blood cell delivery vector of the invention, optionally together with packaging materials therefor and instructions for use. Thus, the present invention further provides a kit comprising a red blood cell delivery vector of the invention, packaging materials therefor and instructions for use comprising the step of causing the agent to be released from the red blood cell delivery vector by applying ultrasound at a frequency and energy to cause disruption of the red blood cell delivery vector but insufficient to cause disruption of unsensitised red blood cells.

In another aspect the present invention provides a kit comprising a red blood cell, an agent, packaging materials therefor and instructions for use in a method comprising the steps of:

(a) presensitising a red blood cell by electrosensitising the cell;

(b) loading the cell with an agent;

(c) electrosensitising the cell; and

(d) causing the agent to be released from the sensitised cell by applying ultrasound at a frequency and energy sufficient to cause disruption of the sensitised cell but insufficient to cause disruption of unsensitised red blood cells,

wherein steps (b) and (c) can be performed in any order.

Also provided is a kit comprising a presensitised red blood cell which is loaded with an agent, packaging materials therefor and instructions for use in a method comprising the steps of:

(a) electrosensitising the cell; and

(b) causing the agent to be released from the sensitised cell by applying ultrasound at a frequency and energy to cause disruption of the sensitised cell but insufficient to cause disruption of unsensitised red blood cells.

Preferably a kit of the invention further comprises polyethylene glycol. Preferably the kit further comprises a liquid selected from a buffer, diluent or other excipient. More preferably the liquid is selected from a saline buffer, a physiological buffer and plasma.

In another aspect the present invention provides a pharmaceutical or physiological composition comprising a red blood cell delivery vector of the invention together with a pharmaceutically or physiologically acceptable carrier or diluent.

The present invention also provides a device for producing a red blood cell delivery vector of the present invention which device comprises:

(a) a flow cell and electrosensitisation means;

(b) a dialysis system,

wherein the flow cell is linked to the dialysis system by connecting means capable of allowing the transfer of red blood cells from the flow cell to the dialysis system and vice versa.

Optionally, the device may comprise more than one flow cell, such as two flow cells. The device may also be connected to a collection device such as a blood bag. In a preferred embodiment, the device is also connected to a chromatography and/or filtration stage so that after the final sensitisation procedure, red blood cells are purified and/or the composition of buffer in which the cells are suspended altered.

### BRIEF DESCRIPTION OF THE DRAWINGS

FIG. 1 demonstrates cell leading levels of an oligonucleotide.

FIG. 2 demonstrates cell loading levels of an antibody.

FIG. 3 shows the results of hypoosmotic dialysis loading of antibody.

FIG. 4 shows the effect of buffer compositions on osmotic loading.

FIG. 5 demonstrates the release of FITC-anti-vWF antibody by ultrasound treatment.

FIG. 6 shows the levels of cell numbers and ultrasound sensitivity in cells in storage.

FIG. 7 shows the retention of payload over 30 days.

### DETAILED DESCRIPTION OF THE INVENTION

Sensitisation

In the method of the present invention, cells are subject to at least two sensitisation steps, one of which must be performed prior to, or concomitant with, the loading step, preferably prior to the loading step. For this reason, the first sensitisation step is referred to herein as a presensitisation step. The purpose of the presensitisation step is to enhance the loading of the agent, although an increase in sensitivity to ultrasound mediated lysis may also be achieved. The additional sensitisation steps may be performed at any stage in the process after the presensitisation step. The purpose of the additional sensitisation step or steps is to increase the sensitivity of the cells to ultrasound.

The term “sensitisation” as used herein, refers to the destabilisation of cells without causing fatal damage to the cells. As used herein, “destabilization” refers to an alteration of a membrane of a cell that makes the cell more susceptible to lysis in vitro or in vivo upon exposure to an energy field such as ultrasound. In one embodiment of the invention, a cell which is destabilized is a cell which is lysed when less than 20%, and preferably less than 5%-10%, or less than 1% of non-sensitised cells are lysed. Destabilisation may be achieved by exposing a cell, such as a red blood cell to an energy field, such as an electric field.

In two particular embodiments that are exemplified herein, a second sensitisation step is carried out either after the presensitisation step but prior to dialysis loading, or after dialysis loading. Further sensitisation steps may be performed as required.

Generally, both of the sensitisation steps and the loading step are temporally separated. For example, cells are typically allowed to rest in buffer, such as PBS/Mg/glucose buffer, for at least 30 mins, preferably at least 60 mins, after a sensitisation step to allow the cells to recover prior to loading or further sensitisation steps. It may be desirable to allow cells to rest for several hours, such as overnight, after the loading step.

We have found that the efficiency of sensitisation for given electrical parameters varies depending on the cell density and it may therefore be necessary to perform a titration of cell density and or electrical parameters to establish the optimum concentration. By way of guidance, we have found that cells sensitised at a density of about 6-8×10⁸ cells/ml had good sensitivity to ultrasound.

Preferably, the sensitisation step comprises an electrosensitisation procedure as described next.

The term “sensitisation procedure” is an event, or events, that occur which destabilizes cells without causing fatal damage to the cells.

As used herein, the term “pre-sensitisation” refers to enhancing the efficiency of loading an agent into a cell, such as a red blood cell, compared to a cell which has not been subjected to pre-sensitisation. In one embodiment, loading efficiency is increased at least two-fold, 5-fold, 1 0-fold, 50- fold, or 100-fold compared to non-pre-sensitised cells. The term “pre-sensitisation” encompasses the destabilisation of cells without causing fatal damage to the cells. As used herein, a pre-sensitisation condition, is any condition to which a cell can be exposed which increases loading efficiency of the cell in comparison to a cell which is not pre-sensitised.

The term “pre-sensitisation procedure” is an event, or events, that occur which destabilizes cells without causing fatal damage to the cells.

Electrosensitisation

The term “electrosensitisation” encompasses the destabilisation of cells without causing fatal damage to the cells. The term “electrosenitisation” as used herein refers to the sensitisation of a cell that occurs upon momentary exposure of the cell to one or more pulses of a high electric field. Electrosensitisation typically involves the use of electric fields which do not possess sufficient energy to electroporate cells. Electroporation, which facilitates the passage of agents into a cell without significant loss of cellular contents or cell viability is well known in the art, and apart from the energy levels involved is similar to electrosensitisation. Cells which are electroporated may become electrosensitised, However, as the term is used in the instant application, electrosensitisation is carried out at energy levels insufficient to electroporate a cell and permit the passage of substances through the cell membrane.

According to this method, a momentary exposure of a cell to one or more pulses at high electric field strength results in membrane destabilisation. The strength of the electric field is adjusted up or down depending upon the resilience or fragility, respectively, of the cells being loaded and the ionic strength of the medium in which the cells are suspended.

Electrosensitisation typically involves the use of electric fields which do not possess sufficient energy to electroporate the cells. Electroporation, which facilitates the passage of agents into the cell without significant loss of cellular contents or cell viability, is well known in the art, and apart from the energy levels involved is similar to electrosensitisation. Indeed, cells which are electroporated become electrosensitised. However, electrosensitisation may be carried out at energy levels which are insufficient to electroporate the cell and permit the passage of substances through the cell wall. Thus, the invention encompasses the use of an electric field for sensitising a red blood cell to ultrasound.

Electroporation has been used in both in vitro and in vivo procedures to introduce foreign material into living cells. With in vitro applications, a sample of live cells is first mixed with the agent of interest and placed between electrodes such as parallel plates. Then, the electrodes apply an electrical field to the cell/implant mixture. Examples of systems that perform in vitro electroporation include the Electro Cell Manipulator ECM600 product, and the Electro Square Porator T820, both supplied by the BTX Division of Genetronics, Inc (see U.S. Pat. No 5,869,326).

These known electroporation techniques (both in vitro and in vivo) function by applying a brief high voltage pulse to electrodes positioned around the treatment region. The electric field generated between the electrodes causes the cell membranes to temporarily become porous, whereupon molecules of the agent of interest enter the cells. In known electroporation applications, this electric field comprises a single square wave pulse on the order of 1 kV/cm, of about 100 μs duration. Such a pulse may be generated, for example, in known applications of the Electro Square Porator T820.

Electrosensitisation may be performed in a manner substantially identical to the procedure followed for electroporation, with the exception that lower electric field strengths may be used, as set forth below.

In a preferred aspect of the present invention, the electric field has a strength of from about 0.1 kV/cm to about 10 kV/cm under in vitro conditions, more from about 1.5 kV/cm to about 4.0 kV/cm under in vitro conditions.

Preferably the electric field has a strength of from about 0.1 kV/cm to about 10 kV/cm under in vivo conditions (see WO97/49450).

Preferably the application of the electric field is in the form of multiple pulses such as double pulses of the same strength and capacitance or sequential pulses of varying strength and/or capacitance. A preferred type of sequential pulsing comprises delivering a pulse of less than 1.5 kV/cm and a capacitance of greater than 5 μF, followed by a pulse of greater than 2.5 kV/cm and a capacitance of less than 2 μF, followed by another pulse of less than 1.5 kV/cm and a capacitance of greater than 5 μF. A particular example is 0.75 kV/cm, 10 μF; 3.625 kV/cm, 1 μF and 0.75 kV/cm, 10 μF.

Preferably the electric pulse is delivered as a waveform selected from an exponential wave form, a square wave form and a modulated wave form.

As used herein, the term “electric pulse” includes one or more pulses at variable capacitance and voltage and including exponential and/or square wave and/or modulated wave forms.

Other electroporation procedures and methods employing electroporation devices are widely used in cell culture, and appropriate instrumentation is well known in the art.

In a particularly preferred embodiment, the following electrosensitisation protocol is used. Cells are suspended in PBS to yield concentrations of about 6-8×10⁸ cells/ml and 0.8 ml aliquots are dispensed into sterile electroporation cuvettes (0.4 cm electrode gap) and retained on ice for 10 min. Cells are then exposed to an electroporation strategy involving delivery of two electric pulses (field strength=3.625 kV/cm at a capacitance of 1 μF) using a BioRad Gene Pulser apparatus. Cells are immediately washed with PBS containing MgCl₂ (4 mM) (PBS/Mg) and retained at room temperature for at least 30 min in the PBS/Mg buffer at a concentration of 7×10⁸ cells/ml to facilitate re-sealing. Optionally, cells are subsequently washed and suspended at a concentration of 7×10⁸ cells/ml in PBS/Mg containing 10 mM glucose (PBS/Mg/glucose) for at least 1 hour.

Loading

As used herein, the term “loading” refers to introducing into a red blood at least one agent. The agent may be loaded by becoming internalised by, affixed to the surface of, or anchored into the plasma membrane of a red blood cell. Loading of a red blood cell with more than one agent may be performed such that the agents are loaded individually (in sequence) or together (simultaneously or concurrently). Loading is generally performed in a separate procedure to the “sensitising” procedure. The agents may be first admixed at the time of contact with the red blood cells or prior to that time.

A “loading procedure” is an event, or events, that occur in the loading module to achieve loading of cells.

According to the present invention, red blood cells are loaded either after the presensitisation procedure or after one or more sensitisation procedures, preferably after the cells have rested. In this embodiment, the loading may be performed by any desired technique.

In a preferred embodiment, the red blood cell may be presensitised by electrosensitisation, and loaded using osmotic shock. If more than one agent is employed, the same or a different technique may be used to load the second agent into the red blood cell.

Preferably the red blood cells of the present invention are presensitised, sensitised and loaded in vitro or ex-vivo.

Preferably loading is carried out by an osmotic shock procedure. The term “osmotic shock” is intended herein to be synonymous with the term “hypotonic dialysis” or “hypoosmotic dialysis”.

A preferred osmotic shock/hypotonic dialysis method is described in the Examples and is based on the method described in Eichler et al., 1986, Res. Exp. Med. 186: 407-412. This preferred method is as follows. Washed red blood cells are suspended in 1 ml of PBS (150 mM NaCl, 5 mM K₂HPO₄/KH₂PO₄; pH 7.4) to obtain a hematocrit of 60%. The suspension is placed in dialysis tubing (molecular weight cutoff 12-14,000; Spectra-Por; prepared as outlined below) and swelling of cells obtained by dialysis against 100 ml of 5 mM K₂HPO₄/KH₂PO₄, pH 7.4 for 90 minutes at 4° C. Resealing is achieved by subsequent dialysis for 15 minutes at 37° C. against 100 ml of PBS containing 10 mM glucose. Cells are then washed in ice cold PBS containing 10 mM glucose using centrifugation.

The term “resealing” encompasses the stabilization of the membrane of a cell by closing pores in the membrane that have previously been opened by some other process, for example, by a loading process such as hypotonic dialysis.

A “resealing procedure” is an event or events that occur to reseal cells.

Other osmotic shock procedures include the method described in U.S. Pat. No. 4,478,824. That method involves incubating a packed red blood cell fraction in a solution containing a compound (such as dimethyl sulphoxide (DMSO) or glycerol) which readily diffuses into and out of cells, rapidly creating a transmembrane osmotic gradient by diluting the suspension of red blood cell in the solution with a near-isotonic aqueous medium. This medium contains an anionic agent to be introduced (such as a phosphorylated inositol) which may be an allosteric effector of haemoglobin, thereby causing diffusion of water into the cells with consequent swelling thereof and increase in permeability of the outer membranes of the cells. This increase in permeability is maintained for a period of time sufficient only to permit transport of the anionic agent into the cells and diffusion of the readily-diffusing compound out of the cells. This method is of limited effectiveness where the desired agent to be loaded into cells is not anionic, or is anionic or polyanionic but is not present in the near-isotonic aqueous medium in sufficient concentration to cause the needed increase in cell permeability without cell destruction.

U.S. Pat. No. 4,931,276 and WO 91/16080 also disclose methods of loading red blood cells with selected agents using an osmotic shock technique. Therefore, these techniques can be used to enable loading of red blood cells in the present invention.

Effective agents which may advantageously be loaded into red blood cells using the modified method provided in U.S. Pat. No. 4,931,276 include peptides, purine analogues, pyrimidine analogues, chemotherapeutic agents and antibiotic agents. These agents frequently present drug delivery problems. Specific compounds include but are not limited to tryptophan, phenylalanine and other water-soluble amino acid compounds. Several derivatives of the unnatural analogues of the nucleic acid bases adenine, guanine, cytosine and thymine are well known as useful therapeutic agents, e.g. 6-mercaptopurine (6MP) and azathioprine, which are commonly used as immunosuppressants and inhibitors of malignant cell growth, and azidothymidine (AZT) and analogues thereof which are useful as anti-viral agents, particularly in the treatment of AIDS. It has been shown that the action of these unnatural base derivatives is dependent on intra-cellular conversion thereof to phosphorylated forms (Chan et al., 1987, Pharmacotherapy, 7: 165;14 177; also Mitsuya et al., 1986, Proc. Natl. Acad. Sci. U.S.A., 83: 1911-1915).

It will be appreciated by one skilled in the art that combinations of methods may be used to facilitate the loading of a red blood cell with agents of interest according to the invention. Likewise, it will be appreciated that a first and second agent, may be loaded concurrently or sequentially, in either order, into a red blood cell in any method of the present invention.

The concentration of agent used in the loading procedure may need to be optimised. For example, we have shown that an FITC-IgG antibody achieves good loading at concentrations of 0.1 mg/ml to 2 mg/ml.

Preferably loading takes place over a period of at least 30 mins, more preferably about 90 mins.

Selective Release using Ultrasound

According to the invention, agents which are loaded into a red blood cell are released from the red blood cell and into their surroundings, in this case at or into the target site, tissue or cell, by the application of ultrasound directed at a target site, tissue and/or cell.

As used herein, the term “ultrasound” refers to a form of energy which consists of mechanical vibrations the frequencies of which are so high they are above the range of human hearing. Lower frequency limit of the ultrasonic spectrum may generally be taken as about 20 kHz. Most diagnostic applications of ultrasound employ frequencies in the range 1 and 15 MHz (from Ultrasonics in Clinical Diagnosis. Edited by PNT Wells, 2nd. Edition, Publ. Churchill Livingstone [Edinburgh, London & N.Y., 1977]. The term “ultrasound” includes diagnostic, therapeutic and focused ultrasound. Diagnostic ultrasound refers to an ultrasound energy source in a range up to about 100 mW/cm² (FDA recommendation). Therapeutic ultrasound refers to an ultrasound energy source in a range up to about 3 to 4 W/cm² (WHO recommendation).

Focused ultrasound (FUS) allows thermal energy to be delivered without an invasive probe (see Morocz et al., 1998 Journal of Magnetic Resonance Imaging Vol.8, No.1, pp.136-142. Another form of focused ultrasound is high intensity focused ultrasound (HIFU) which is reviewed by Moussatov et al. in Ultrasonics, 1998 Vol.36, No.8, pp.893-900 and TranHuuHue et al. in Acustica, 1997, Vol.83, No.6, pp. 1103-1106.

Preferably, a combination of diagnostic ultrasound and a therapeutic ultrasound is employed.

Preferably the ultrasound is applied to a target cell or target tissue with sufficient strength to disrupt loaded and sensitised red blood cells but without damaging the target tissue or surrounding tissues. In the context of the present invention, the term “damage or damaging” does not include a transient permeabilisation of the target site by the ultrasound energy source. Such a permeabilisation may facilitate uptake of the released payload at the target site.

Preferably the exposure to an ultrasound energy source is at a power density of from about 0.05 to about 100 Wcm⁻². Even more preferably, the exposure to an ultrasound energy source is at a power density of from about 1 to about 15 Wcm⁻².

Preferably the exposure to an ultrasound energy source is at a frequency of from about 0.015 to about 10.0 MHz. More preferably the exposure to an ultrasound energy source is at a frequency of from about 0.02 to about 5.0 MHz.

Preferably the exposure is for periods of from about 10 milliseconds to about 60 minutes. More preferably the exposure is for periods of from about 1 second to about 5 minutes.

Particularly preferably the patient is exposed to an ultrasound energy source at an acoustic power density of from about 0.05 Wcm⁻² to about 10 Wcm⁻² with a frequency ranging from about 0.015 to about 10 MHz (see WO 98/52609).

Use of ultrasound is advantageous as, like light, it can be focused accurately on a target. Moreover, ultrasound is advantageous as it has a broader degree of three-dimensional focus than a light energy source and is better suited to whole-tissue penetration (such as but not limited to a lobe of the liver) or whole organ (such as but not limited to the entire liver or an entire muscle, such as the heart) delivery of agents according to the present invention. In addition, ultrasound may induce a transient permeabilisation of the target site so that uptake of a released payload is facilitated at the target site. Another important advantage is that ultrasound is a non-invasive stimulus which is used in a wide variety of diagnostic and therapeutic applications. By way of example, ultrasound is well known in medical imaging techniques and, additionally, in orthopaedic therapy. Furthermore, instruments suitable for the application of ultrasound to a subject vertebrate are widely available and their use is well known in the art.

In methods of the invention, release of the agent is effected by exposure of red blood cells either in vitro or ex-vivo to an effective amount of a diagnostic ultrasound energy source or a therapeutic ultrasound energy source as described in U.S. Pat. No. 5,558,092 and WO94/28873. The agent, which is released from a red blood cell for use in the present invention may be referred to as the “payload” of that cell.

Preferably the agent is released from the red blood cell by treatment of a target site, tissue or cell with ultrasound.

The selective release of the agent at the target site can be determined by observing a) the amount which has been released at the target site, tissue or cell and b) its effect on the target site, tissue or cell, the latter determining whether its delivery should increase, decrease or be discontinued.

Blood Cells

In one embodiment of the present invention, the red blood cells which may be loaded and administered to a vertebrate according to the invention are ideally obtained from the intended recipient individual prior to the procedure so as to ensure complete immunocompatibility. Alternatively, cells are obtained from a second individual of the same species as the recipient; in such a case, the second individual must share the blood type of the intended recipient or must have an immuno-neutral blood type, such as type O in humans. Alternatively, the red blood cell may have its immunological determinants masked by a substance such as PEG and/or modified, for example by one or more enzymes.

As used herein, the term “red blood cell” refers to a living, enucleate red blood cell (i.e., a mature erythrocyte) of a vertebrate.

Preferably the red blood cell is a mammalian red blood cell, advantageously a human red blood cell. As used herein, the term “mammal” refers to a member of the class Mammalia including, but not limited to, a rodent, lagomorph, pig or primate. Preferably, the mammal is a human.

As used herein the term “introducing” includes but is not limited to the administration of a red blood cell and/or an agent into a vertebrate.

As used herein in reference to administration of an agent to a vertebrate, the term “introducing” includes but is not limited to causing the agent to enter the circulatory system of the vertebrate by transfusion or to infusing an agent to a target site. It is contemplated that a hollow needle, such as a hypodermic needle or cannula, is inserted through the wall of a blood vessel (e.g., a vein or artery) and the red blood cell is either injected using applied pressure or allowed to diffuse or otherwise migrate into the blood vessel. It is understood that the diameter of the needle is sufficiently large and the pressure sufficiently light to avoid damage of the cell by shear forces. Preferably, introduction of a red blood cell into a vertebrate in a method of the invention is intra-arterial or intravenous. Methods of blood cell transfusion are well known in the art.

As used herein, the term “red blood cell delivery vector” means a red blood cell that has been electrosensitised and loaded with one or more agents according to the methods of the invention and can be used to deliver the agent to a vertebrate. The red blood cell delivery vector is typically made to release the agent at a site of interest in the vertebrate using ultrasound as described above.

Agent

As used herein, the term “agent” includes but is not limited to an atom or molecule, wherein a molecule may be inorganic or organic, a biological effector molecule and/or a nucleic acid encoding an agent such as a biological effector molecule, a protein, a polypeptide, a peptide, a nucleic acid, a virus, a virus-like particle, a nucleotide, a ribonucleotide, a synthetic analogue of a nucleotide, a synthetic analogue of a ribonucleotide, a modified nucleotide, a modified ribonucleotide, an amino acid, an amino acid analogue, a modified amino acid, a modified amino acid analogue, a steroid, a proteoglycan, a lipid, a carbohydrate, an oligosaccharide and a glycoprotein. An agent may be in solution or in suspension (e.g., in crystalline, colloidal or other particulate form).

As used herein, the term “biological effector molecule” refers to an agent that has activity in a biological system, including, but not limited to, a protein, polypeptide or peptide including, but not limited to, a structural protein, an enzyme, a cytokine (such as an interferon and/or an interleukin) an antibiotic, a polyclonal or monoclonal antibody, or an effective part thereof, such as an Fv fragment, a single chain Fv (scFv), a Fab or a F(ab′)₂, which antibody or part thereof may be natural, synthetic or humanised, a peptide hormone, a receptor, a signalling molecule or other protein; a nucleic acid, as defined below, including, but not limited to, an oligonucleotide or modified oligonucleotide, an antisense oligonucleotide or modified antisense oligonucleotide, cDNA, genomic DNA, an artificial or natural chromosome (e.g. a yeast artificial chromosome) or a part thereof, RNA, including mRNA, tRNA, rRNA or a ribozyme, or a peptide nucleic acid (PNA); a virus or virus-like particles; a nucleotide or ribonucleotide or synthetic analogue thereof, which may be modified or unmodified; an amino acid or analogue thereof, which may be modified or unmodified; a non-peptide (e.g., steroid) hormone; a proteoglycan; a lipid; an oligosaccharide; a glycoprotein; or a carbohydrate. If the biological effector molecule is a polypeptide, it may be loaded directly into a red blood cell of the invention; alternatively, a nucleic acid molecule bearing a sequence encoding the polypeptide, which sequence is operatively linked to transcriptional and translational regulatory elements active in a cell at the target site, may be loaded. Small molecules, including inorganic and organic chemicals, are also of use in the present invention.

Particularly useful classes of biological effector molecules include, but are not limited to, antibiotics, anti-inflammatory drugs, angiogenic or vasoactive agents, growth factors and cytotoxic agents (e.g., tumour suppressers). Cytotoxic agents of use in the invention include, but are not limited to, diptheria toxin, Pseudomonas exotoxin, cholera toxin, pertussis toxin, and the prodrugs peptidyl-p-phenylenediamine-mustard, benzoic acid mustard glutamates, ganciclovir, 6-methoxypurine arabinonucleoside (araM), 5-fluorocytosine, glucose, hypoxanthine, methotrexate-alanine, N-[4-(a-D-galactopyranosyl) benyloxycarbonyl]-daunorubicin, amygdalin, azobenzene mustards, glutamyl p-phenylenediamine mustard, phenolmustard-glucuronide, epirubicin-glucuronide, vinca-cephalosporin,phenylenediamine mustard-cephalosporin, nitrogen-mustard-cephalosporin, phenolmustard phosphate, doxorubicin phosphate, mitomycin phosphate, etoposide phosphate, palytoxin-4-hydroxyphenyl-acetamide, doxorubicin-phenoxyacetamide, melphalan-phenoxyacetamide, cyclophosphamide, ifosfamide or analogues thereof. If a prodrug is loaded in inactive form, a second biological effector molecule may be loaded into the red blood cell of the present invention. Such a second biological effector molecule is usefully an activating polypeptide which converts the inactive prodrug to active drug form, and which activating polypeptide is selected from the group that includes, but is not limited to, viral thymidine kinase (encoded by Genbank Accession No. J02224), carboxypeptidase A (encoded by Genbank Accession No. M27717), α-galactosidase (encoded by Genbank Accession No. M13571), β-glucuronidase (encoded by Genbank Accession No. M15182), alkaline phosphatase (encoded by Genbank Accession No. J03252 J03512), or cytochrome P-450 (encoded by Genbank Accession No. D00003 N00003), plasmin, carboxypeptidase G2, cytosine deaminase, glucose oxidase, xanthine oxidase, β-glucosidase, azoreductase, t-gutamyl transferase, β-lactamase, or penicillin amidase. Preferably, the polypeptide capable of activating a prodrug is DT diaphorase. Either the polypeptide or the gene encoding it may be loaded; if the latter, both the prodrug and the activating polypeptide may be encoded by genes on the same recombinant nucleic acid construct.

Preferably the biological effector molecule is selected from the group consisting of a protein, a polypeptide, a peptide, a nucleic acid, a virus, a virus-like particle, a nucleotide, a ribonucleotide, a synthetic analogue of a nucleotide, a synthetic analogue of a ribonucleotide, a modified nucleotide, a modified ribonucleotide, an amino acid, an amino acid analogue, a modified amino acid, a modified amino acid analogue, a steroid, a proteoglycan, a lipid and a carbohydrate or a combination thereof (e.g., chromosomal material comprising both protein and DNA components or a pair or set of effectors, wherein one or more convert another to active form, for example catalytically).

The present invention advantageously employs agents which are not able to diffuse through an intact erythrocyte cell wall by passive or active means. However, the delivery of agents which diffuse at a certain rate through the erythrocyte cell wall is contemplated, particularly where increased delivery of the agent at a particular time or location is desirable. Increased delivery may be achieved by ultrasound administration at the appropriate time or location.

Nucleic Acid

A nucleic acid of use in the invention may comprise a viral or non-viral DNA or RNA vector, where non-viral vectors include, but are not limited to, plasmids, linear nucleic acid molecules, artificial chromosomes and episomal vectors. Expression of heterologous genes has been observed after injection of plasmid DNA into muscle (Wolff J. A. et al., 1990, Science, 247: 1465-1468; Carson D. A. et al., U.S. Pat. No. 5,580,859), thyroid (Sykes et al., 1994, Human Gene Ther., 5: 837-844), melanoma (Vile et al., 1993, Cancer Res., 53: 962-967), skin (Hengge et al., 1995, Nature Genet., 10: 161-166), liver (Hickman et al., 1994, Human Gene Therapy, 5: 1477-1483) and after exposure of airway epithelium (Meyer et al., 1995, Gene Therapy, 2: 450-460).

As used herein, the term “nucleic acid” is defined to encompass DNA and RNA or both synthetic and natural origin which DNA or RNA may contain modified or unmodified deoxy-or dideoxy- nucleotides or ribonucleotides or analogues thereof. The nucleic acid may exist as single- or double-stranded DNA or RNA, an RNA/DNA heteroduplex or an RNA/DNA copolymer, wherein the term “copolymer” refers to a single nucleic acid strand that comprises both ribonucleotides and deoxyribonucleotides.

The term “synthetic”, as used herein, is defined as that which is produced by in vitro chemical or enzymatic synthesis.

Therapeutic nucleic acid sequences useful according to the methods of the invention include those encoding receptors, enzymes, ligands, regulatory factors, and structural proteins. Therapeutic nucleic acid sequences also include sequences encoding nuclear proteins, cytoplasmic proteins, mitochondrial proteins, secreted proteins, plasmalemma-associated proteins, serum proteins, viral antigens, bacterial antigens, protozoal antigens and parasitic antigens. Therapeutic nucleic acid sequences useful according to the invention also include sequences encoding proteins, lipoproteins, glycoproteins, phosphoproteins and nucleic acids (e.g., RNAs such as ribozymes or antistnse nucleic acids). Ribozymes of the hammerhead class are the smallest known, and lend themselves both to in vitro synthesis and delivery to cells (summarised by Sullivan, 1994, J. Invest. Dermatol., 103: 85S-98S; Usman et al., 1996, Curr. Opin. Struct. Biol., 6: 527-533). Proteins or polypeptides which can be expressed by nucleic acid molecules delivered according to the present invention include hormones, growth factors, neurotransmitters, enzymes, clotting factors, apolipoproteins, receptors, drugs, oncogenes, tumour antigens, tumour suppressers, structural proteins, viral antigens, parasitic antigens and bacterial antigens. The compounds which can be incorporated are only limited by the availability of the nucleic acid sequence encoding a given protein or polypeptide. One skilled in the art will readily recognise that as more proteins and polypeptides become identified, their corresponding genes can be cloned into the gene expression vector(s) of choice, administered to a tissue of a recipient patient or other vertebrate, and expressed in that tissue.

Delivery of Agents

The method of the present invention is useful for the delivery of agents to a selected site in a vertebrate body, whether an organ, part of an organ or otherwise, in the presence or absence of specific targeting means. This is achieved, as set out above, by the selective disruption by ultrasound at the selected target site of electrosensitised red blood cells loaded with the agent of choice.

As used herein, the term “target site” is the site to which the delivery vehicle or cell loaded with a biological effector molecule will be delivered. Agents useful for use in the present invention are set out above. Preferred agents include those useful for imaging of tissues in vivo or ex vivo. For example, imaging agents, such as antibodies which are specific for defined molecules, tissues or cells in an organism, may be used to image specific parts of the body by releasing them at a desired location using ultrasound. This allows imaging agents which are not completely specific for the desired target, and which might otherwise lead to more general imaging throughout the organism, to be used to image defined tissues or structures. For example, an antibody which is capable of imaging endothelial tissue may be used to image liver vasculature by releasing the antibody selectively in the liver by applying ultrasound thereto.

As used herein, an “imaging agent” is an agent which may be detected, whether in vitro or in vivo in the context of a tissue, organ or organism in which the agent is located.

Kits

The invention also encompasses a number of kits. Some of the kits comprise partially or fully treated red blood cells. Other kits provide a red blood cell, an agent and packaging materials therefor together with instructions for carrying out the methods of the invention.

A kit designed for the easy delivery of an agent to a recipient vertebrate, whether in a research of clinical setting, is encompassed by the present invention. A kit takes one of several forms, as follows:

A kit for the delivery of an agent to a subject vertebrate comprises red blood cells and the agent and instructions for performing the method of the present invention. Alternatively, the red blood cells are supplied loaded with the agent for convenience of use by the purchaser. In the latter case, the cells are supplied in sensitised form, ready for rapid use or presensitised and loaded but needing a final sensitisation step.

The cells of the kit are typically species-specific to the vertebrate of interest, such as a primate, including a human, canine, rodent, pig or other, as desired; in other words, the cells are of like species with the intended recipient. The cells of the kit are, additionally, specific to the blood type of the intended recipient organism, as needed. Optionally, the kit comprises one or more buffers for cell sensitisation, washing, resuspension, dilution and/or administration to a vertebrate. Appropriate buffers are selected from the group that includes low ionic strength saline, physiological buffers such as PBS or Ringer's solution, cell culture medium and blood plasma or lymphatic fluid. The kit additionally comprises packaging materials (such as tubes, vials, bottles, or sealed bags or pouches) for each individual component and an outer packaging, such as a box, canister or cooler, which contains all of the components of the kit. The kit is shipped refrigerated. Optionally, non-cellular components are supplied at room temperature or frozen, as needed to maintain their activity during storage and shipping. They may be in liquid or dry (i.e., powder) form.

A second kit of the invention comprises an agent such as a biological effector molecule, instructions for performing the method of the present invention and, optionally a sensitising device and buffers therefor (e.g., saline or other physiological salt buffer, culture medium, plasma or lymphatic fluid). In addition, the kit contains appropriate packaging materials, as described above. The individual components may be supplied in liquid or dry (i.e., powder) form, and may be at room temperature, refrigerated or frozen as needed to maintain their activity during storage and shipping. Red blood cells for use with this kit may be obtained independently (for example, they may be harvested from the intended recipient vertebrate).

A preferred aspect of the invention is a kit comprising a red blood cell which is loaded with an agent, and packaging materials therefor.

Preferably, a kit as described above further comprises an apparatus for applying the sensitising procedure.

Preferably a kit of the invention further comprises polyethylene glycol. Preferably the kit further comprises a liquid selected from a buffer, diluent or other excipient. More preferably the liquid is selected from a saline buffer, a physiological buffer and plasma.

Another aspect of the invention is a physiological composition comprising a red blood cell delivery vector of the invention comprising a biological effector molecule admixed with a physiologically compatible buffer or pharmaceutically acceptable carrier or diluent. As used herein, the term “physiologically compatible buffer” or “physiological buffer” or “pharmaceutically acceptable carrier or diluent” is defined as a liquid composition which, when placed in contact with living cells, permits the cells to remain alive over a period of minutes, hours or days. As such, a physiological buffer is substantially isotonic with the cell, such that cell volume does not change more than 20% due to differences in internal and external ionic strength. Non-limiting examples of physiologically compatible buffers or physiological buffers include dilute saline, which may be buffered (e.g., Hanks' buffered saline or phosphate buffered saline), or other physiological salts (e.g., Ringer's solution), dilute glucose, sucrose or other sugar, dilute glycerol with- or without salts or sugars, cell culture media as are known in the art, serum and plasma.

Preferably, the red blood cell of the physiological composition is a human cell.

### EXAMPLES

### Example 1

### Loading of RBC with Oligonucleotides

In the following example three protocols for the loading and sensitisation of red blood cells (RBC/erythrocytes) are demonstrated and compared.

The first procedure demonstrates loading and sensitisation of red blood cells by exponential wave electric or square wave electric field pulse loading. Such electric pulses used for loading or sensitisation are abbreviated as ES. The second procedure consists of loading and sensitisation of red blood cells by a combination of electrosensitisation followed by hypoosmotic dialysis loading (HD, dialysis or osmotic loading). The combination is abbreviated as ES+HD. The third procedure consists of loading and sensitisation of red blood cells by electrosensitisation, followed by hypoosmotic dialysis, overnight rest and further treatment of the cells by electrosensitisation. This combination is abbreviated as ES+HD+ES.

In the first procedure, red blood cells are loaded with an oligonucleotide by a conventional electroporation procedure, as described in the prior art, using exponential wave electric pulses. Briefly, human blood was harvested by venipuncture and washed twice in PBS (phosphate buffered saline) by centrifugation. Cells were suspended in PBS containing 60 μg/ml of a random 30-mer FITC-labelled oligonucleotide to yield concentrations of 3.5×10⁸ cells/ml and 0.8 ml aliquots were dispensed into sterile electroporation cuvettes (0.4 cm electrode gap) and retained on ice for 10 min. Cells were then exposed to an electroporation strategy involving delivery of two electric pulses (field strength=3.625 kV/cm at a capacitance of 1 μF) using a BioRad Gene Pulser apparatus. Cells were immediately washed with PBS containing MgCl₂ (4 mM) (PBS/Mg) and retained at room temperature for 30 min in the PBS/Mg buffer to facilitate re-sealing. Cells were subsequently washed and suspended at a concentration Of between 7 and 14×10⁸ cells/ml in PBS/Mg containing 10 mM glucose (PBS/Mg/glucose) for at least 1 hour.

The second procedure employed is essentially as described in our copending UK patent application 9917416.1. Briefly, 10 ml of peripheral venous blood is collected by venipuncture, into lithium heparin anticoagulant containing tubes, and mixed gently. The whole blood is then poured into a polypropylene tube and centrifuged at 300 g for 15 min at room temperature. The plasma and white blood cells (buffy coat) are removed.

1x phosphate buffered saline (PBS, made from Oxoid tablets code BR14a pH7.3) is added and the cells centrifuged at 700 g for 5 min. The supernatant is removed and the pellet of remaining cells resuspended in ice cold 1xPBS. The spin/wash procedure is then repeated once, and cells are suspended in ice-cold PBS at 6×10⁸ cells/ml.

Cells are then electrosensitised by dispensing 800 μl of the RBC into sterile electroporation cuvettes, and placed on ice. To electrosensitise the cells, they are exposed to an electric field at 3.625 kV/cm, 1 μF (2 pulses), in the absence of payload. The RBCs are then removed, and pooled in polypropylene tubes.

Cells are centrifuged once at 700 g for 5 min at room temperature (RT). The cells may be diluted in PBS/MgCl₂ (4mM). Cells are then resuspended in PBS/MgCl₂, and centrifuged at 700 g for 5 min, twice. Finally, cells are resuspended in PBS/MgCl₂, at approximately 7×10⁸ c/ml, and rested for 30 min at room temperature.

Cells are then loaded with oligonucleotide by hypoosmotic dialysis, according to a protocol adapted from Eichler et al., (1986) Clin. Pharmacol. Ther. 40:300-303. The following protocol is followed:

1 BUFFERS:

Stock potassium phosphate buffer:

5mM K₂HPO₄3H₂O (FW 228.2 g)→1.141 g/L

5mM KH₂PO₄(MW136.1 g)→0.68 g/L

Stored at 4° C.

Mix as follows:

For a pH7.4 K₂H/KH₂ phosphate buffer→approx. 6.1:3.9 parts

Mix the 2 stock solutions as and when required

Buffer #1 (isoosmotic PBS):

pH7.4 K₂H/KH₂ phosphate buffer

150 mM NaCl→8.76 g/L

Check and adjust pH (1M NaOH)

Buffer #2 (dialysis buffer):

pH7.4 K₂H/KH₂ phosphate buffer

Check and adjust pH (1M NaOH)

Buffer #3 (resealing buffer)

pH7.4 K₂H/KH₂ phosphate buffer

150 mM NaCl→8.76 g/L

10 mM glucose→1.8 g/L

Check and adjust pH (1M NaOH)

2 SpectraPor dialysis tubing:

1 The 12-14 kDa MW cut off tubing, 0.32 ml/cm, is used.

2 Preparation: heat at 80° C./30min in 1 mM EDTA/2% sodium bicarbonate (Sigma). Rinse well, inside and outside, with ddH₂O.

3 Wash inside and outside with Buffer #1

4 Store submerged in a small amount of Buffer #1 if not used immediately.

3 RBC PREPARATION:

1 Electrosensitised, rested RBC are washed in PBS twice at 700 g for 5 min.

2 For the final wash, cells are washed in buffer #1

3 The cells are manipulated as a suspension of packed cells following removal of final wash supernatants after centrifugation.

4 CELL VOLUME IN TUBING:

1 Protocol recommends 60% haematocrit (HCT). The suspension of packed cells is approximately 75% HCT and is diluted accordingly.

2 Mix cells with the oligonucleotide and buffer #1, to give required final oligonucleotide concentration and volume.

5 DIALYSIS:

1 The tubing is clipped to ensure that the surface area remains constant for the volume of cells.

2 Dialyse RBC (packed cell volume in buffer #1) against buffer #2 for 90 min at 4° C.

3 Place membranes in 100-200 ml buffer #2, (ensure that the membrane is immersed) in glass beaker with magnetic flea.

4 Place this beaker within another beaker, which contains ice, on the magnetic stirrer, cover with silver foil.

6 Warm up an aliquot of buffer #3 to 37° C.

7 Remove dialysis buffer, replace with the warm resealing buffer #3.

8 Place beaker with dialysis tubing and buffer #3 into a larger beaker anchored by water at 37° C., cover and leave for 15 min.

9 Harvest cells into 12 ml polypropylene tubes.

10 Wash x3 in ice cold resealing buffer #3 at 300 g, 10 min 4° C.

11 Wash x1 in PBS/Mg/glucose and spin at 700 g, 5 min 4° C.

12 Count cells and resuspend at 7×10⁸c/ml, in PBS/Mg/glucose.

13 Store at 4° C. overnight.

In the present example, dialysis is performed in the presence of 10 μg of oligonucleotide per ml of cells. Cells are suspended at 7×10⁸ cells/ml.

In the third procedure, cells are prepared as described for the second procedure, but exposed to an additional electrosensitisation step after loading by dialysis, according to the following protocol.

1 Following overnight storage, wash RBC once in PBS 700 g, 5 min 4° C.

2 Count cells and resuspend at 6×10⁸c/ml, in ice cold PBS.

3 Dispense 800 μl of the RBC into sterile electroporation cuvettes (0.4 cm gap).

4 Place on ice.

5 To electrosensitise: double pulse at 3.625 kV/cm, 1 μF.

6 Harvest the RBC, pool in a polypropylene tube.

7 Centrifuge once at 700 g for 5 min room temperature (RT). The cells may be diluted in PBS/MgCl₂(4 mM).

8 Resuspend in PBS/MgCl₂, centrifuge at 700 g for 5 min.

9 Repeat step 6

10 Resuspend in PBS/MgCl₂, at approximately 7×10⁸c/ml.

11 Rest the cells for 30 min at RT.

12 Centrifuge once at 700 g for 5 min room temperature (RT). The cells may be diluted in PBS/MgCl₂/glucose.

13 Resuspend the cells in PBS/MgCl₂/glucose, centrifuge at 700 g for 5 min.

14 Repeat step 13.

15 Resuspend cells in PBS/MgCl₂/glucose at 7×10⁸c/ml.

16 Rest the cells in PBS/MgCl₂/glucose for 60 min.

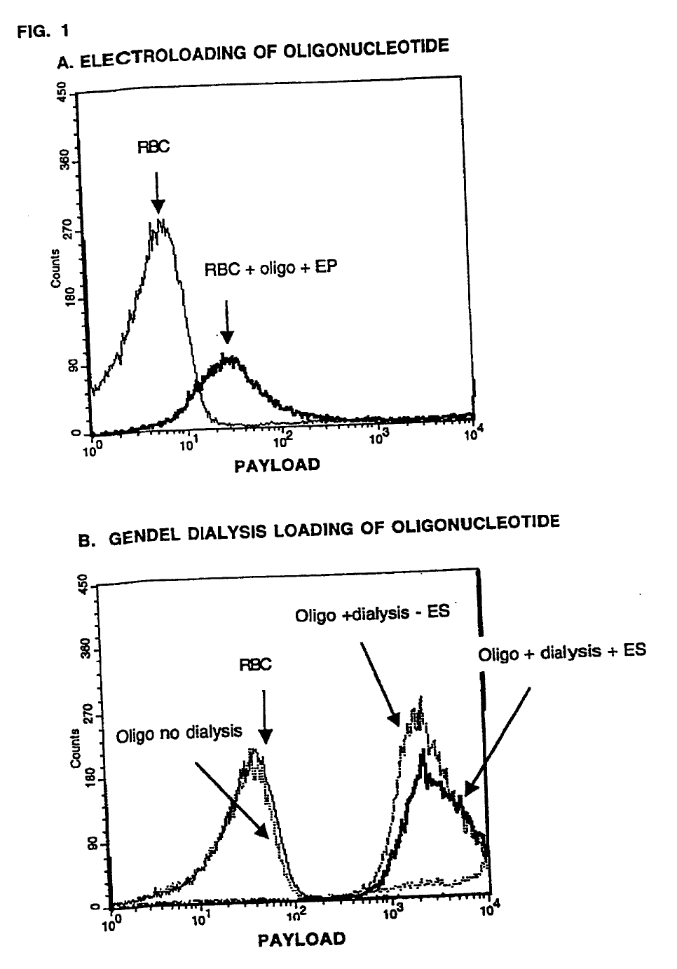

Cells prepared according to all three procedures were analysed to determine cell loading levels and subjected to ultrasound disruption. The results are shown in FIG. 1.

For the electroporated cells, shown in FIG. 1A, the oligonucleotide (oligo) did not bind non-specifically to RBC as the mean fluorescence intensity (MFI) was 1. The MFI is defined as the ratio of fluorescence associated with loaded cells divided by the fluorescence associated with non-specific binding. The electroloaded cells have an increase in fluorescence, with an MFI of 5.6.

Ultrasound sensitivity was measured at 0.75W/cm², 3 MHz, 30 sec. 0% of control cells lysed, compared with 20% of the electroporated cells.

For cells loaded by electrosensitisation followed by hypoosmotic dialysis (ES+HD), non-specific binding is negligible (MFI=1). Loading by ES+HD results in a marked increase in fluorescence with an MFI of 80. Incorporation of the second electrosensitisation step (ES+HD+ES) has little effect on the level of fluorescence with an MFI of 93 indicating retention of the payload during the procedure.

Ultrasound sensitivity is measured at 3W/cm², 1 MHz, 35 sec in a TMM (tissue mimicking medium). Cells subjected to only a single electrosensitisation and dialysis procedure (ES+HD) show 28% lysis, whilst cells subjected to the additional electrosensitisation step (ES+HD+ES) show 89% lysis.

### Example 2

### Loading of RBC with Antibodies

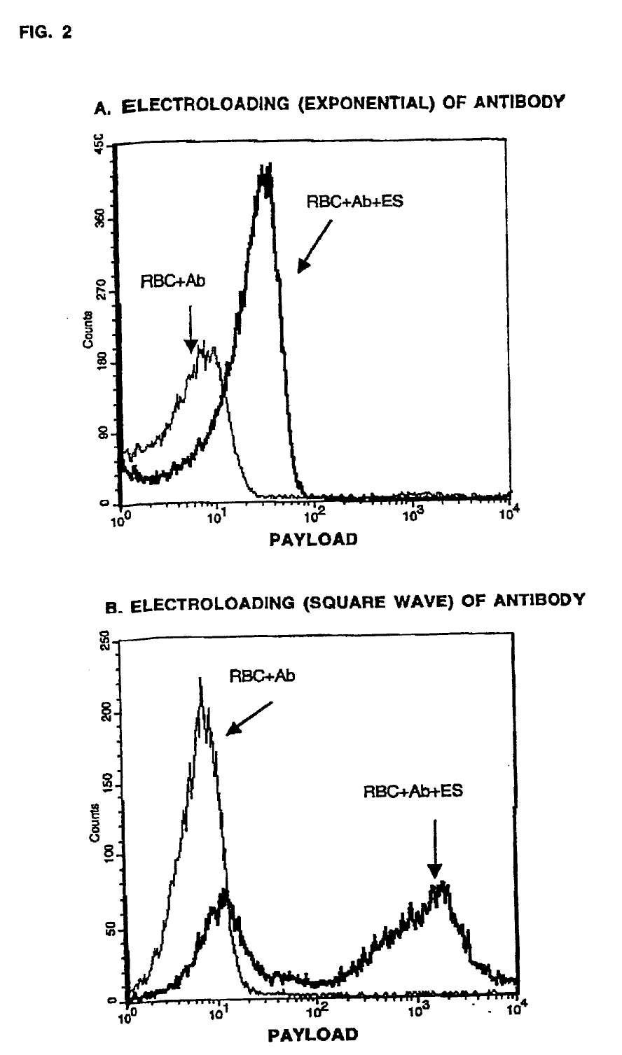

For comparative purposes, antibodies are loaded into RBC by electroporation. The antibody used is a FITC-conjugated anti-vWF antibody (Sigma). The results are shown in FIG. 2.

In a first procedure (FIG. 2A), cells are loaded as described in Example 1 by exposing 3.5×10⁸ cells/ml to 3 pulses of an exponential wave electric field in the presence of antibody at 0.25 mg/ml. An MFI of 3.98 is observed, with 100% ultrasound sensitivity. However, in this procedure the cell recovery is low, approximately 11 %.

In a second procedure (FIG. 2B), 10 pulses of a square wave electric field are used, in the presence of 0.5 mg/ml antibody (the conditions are optimised for each protocol). Two peaks are seen in the cell population after loading, with an MFI of 1.73 and 184.8. 40% of the cells are recovered, of which 97% are ultrasound sensitive when exposed to ultrasound at an energy of 1.25 W/cm² using a 3 MHz probe for 30s. 0.005 pg antibody per cell was recovered

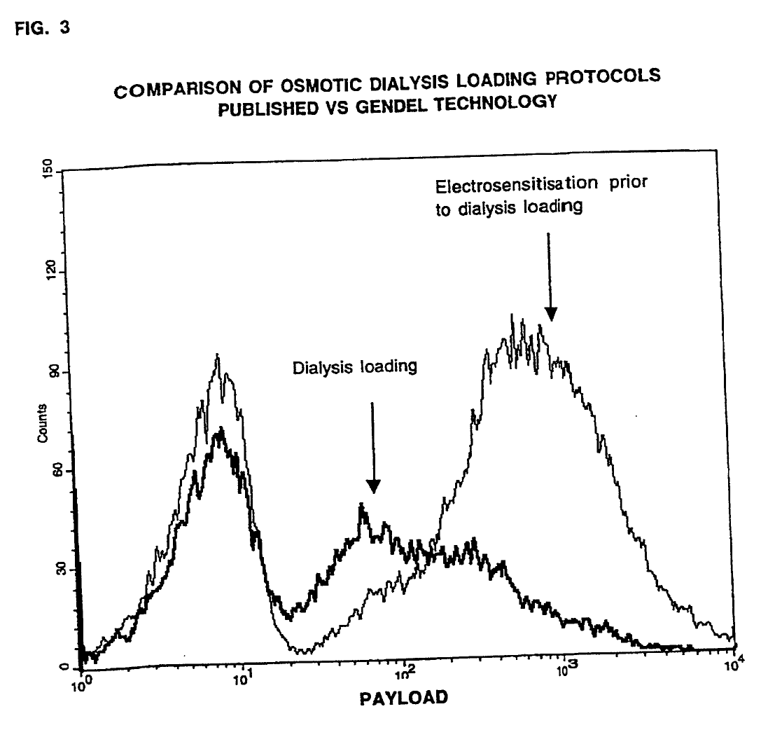

FIG. 3 shows the results of hypoosmotic dialysis loading of antibody according to the procedure of Eichler et al. and of the present invention (see Example 1). The relative MFI is 15.2 in the absence of electrosensitisation (HD loading alone) compared to an MFI of 61.7 in the presence of electrosensitisation (ES+HD). This demonstrates a dramatic increase in loading.

In both dialysis protocols, 80 to 90% of the cells are recovered. Ultrasound sensitivity is about 30% in the absence of a second sensitisation step (ES+HD). Following the second sensitisation step (ES+HD+ES) there is no apparent leakage/loss of the payload and about 90-100% of the cells are ultrasound sensitive (see table in Example 6). 0.22 pg antibody/cell was recovered.

### Example 3

### Resealing Buffers

An alternative resealing buffer is tested in order to assess any impact on the performance of the method according to the invention. The buffer of Bax et al., (1999) Clinical Science 96:171-178, is compared to the buffer adapted from Eichler et aL as used in Example 1 (ES+HD).

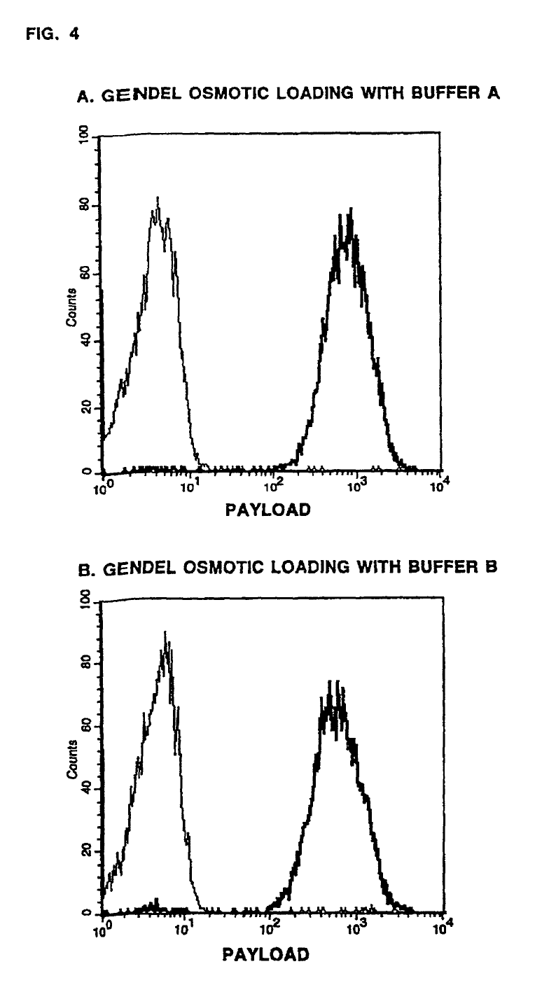

As can be seen from FIG. 4, the performance of the two buffers is almost identical. In both cases, cells were loaded with FITC conjugated anti-vWF antibody as described in Example 2, and subjected to a second electrosensitisation procedure in accordance with the present invention.

The mean fluorescence intensities observed for the Eichler and Bax buffers are 186 and 126 respectively. Observed cell losses are 27% and 16%.

Ultrasound sensitivities are measured in a TMM, at 3 W/cm², for 35 seconds; cell lysis of 68% and 72% is observed, respectively.

### Example 4

### Release of Payload by Ultrasound

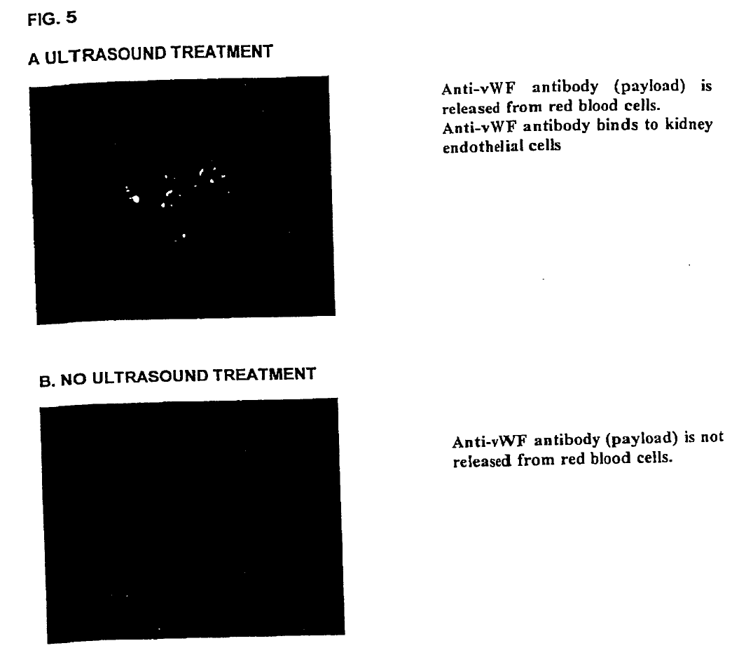

The release of FITC-conjugated antibody by ultrasound in PBS-perfused kidney tissue is shown in FIG. 5. RBC are loaded with FITC-anti-vWF antibody as described in Example 2 above, in accordance with the present invention, and administered to PBS-perfused kidneys, according to the following protocol:

Perfuse the rat through the heart with PBS/EDTA until the kidney is clear of blood

Remove the dorsal aorta from the heart and insert a gavage needle into the vessel. Tie the needle to the dorsal aorta using suture.

Close the dorsal aorta and posterior vena cava just after the junction leading to the kidney.

Close the left adrenal artery and vein and both anterior mesenteric and coeliac arteries

Close the ureter and the left iliolumbar artery and vein.

Create an exit point by inserting a gavage needle into the vena cava just before the liver. Tie the needle using suture.

Flush with 10 ml PBS/4mM Mg/10 mM glucose and check for any leakage.

Block the exit point by inserting 2 ml syringe into the gavage needle.

Load 1 ml of 7×10⁸ cells /ml through the dorsal aorta into the kidney.

Treat with U/S using 1 MHz probe.

Incubate the treated kidney for one hour

Remove the 2 ml syringe and flush through with 2 ml PBS/Mg/glucose

Collect the flush through for cell counting and ELISA

Flush with 50 ml of PBS/EDTA

Flush with 20 ml of 4% neutral buffered formalin (NBF)

Remove the U/S-treated kidney and cut it into two half's and fix in NBF

Prepare tissue sections (12 μm)and stain using Vectastain ABC kit (Vecta Labs) as outlined in the manufacturer's instructions.

Preparation of RBC: dialysed and electrosensitised (ES+HD+ES), antibody loaded erythrocytes:

Rat 1 No ultrasound treatment

Rat 2 Ultrasound treatment at 3 W/cm² for 40 seconds.

Kidney endothelial cells in glomeruli are labelled by the FITC conjugated anti-vWF antibody after ultrasound treatment to release the antibody, as shown in FIG. 5A. In the absence of ultrasound treatment, no staining is observed (FIG. 5B).

### Example 5

### Stability of Loaded Cells

RBC are loaded by dialysis according to the present invention (ES+HD+ES), as described in Examples 1 and 2, with FITC-conjugated antibody. Following the second sensitisation, cells are stored at 7×10⁸ cells/ml in SAGM buffer (Blood transfusion service buffer, obtainable from Baxter Health Care). Cells are stored with maximal exclusion of air at 4° C. Maintenance of ultrasound sensitivity, cell numbers and payload are assessed over a period of 35 days.

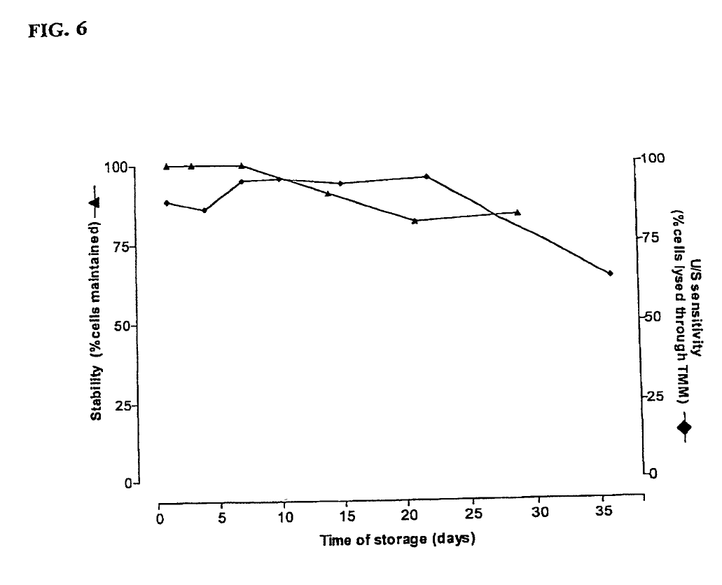

FIG. 6 shows the levels of cell numbers and ultrasound sensitivity in cells on storage. Ultrasound sensitivity, measured at 3 W/cm², 35 sec, in a TMM, is maintained at or above the starting level of 90% for 25 days, and falls to about 65% after 35 days. Cell numbers are stable over a 30 day period.

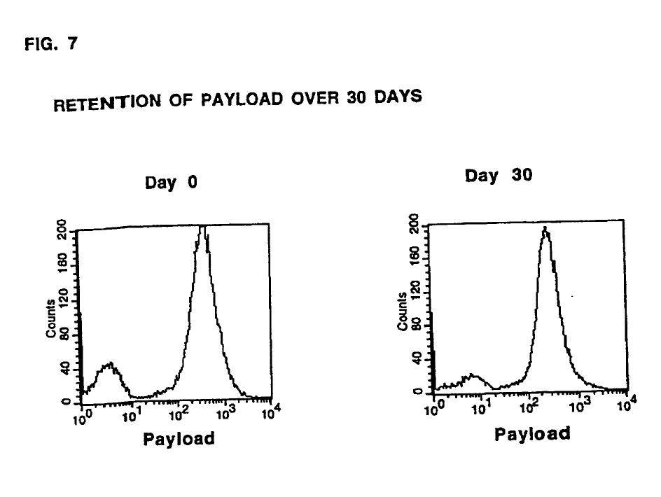

FIG. 7 shows the retention of payload over 30 days under identical conditions to the above. No loss of payload is observed.

### Example 6

### Comparison of different sequences of sensitisation and osmotic loading steps

The hypoosmotic dialysis loading protocol described in Example 1 is performed in two different configurations to determine the effect on loading efficiency and susceptibility to ultrasound mediated lysis when the loading step is performed before the second sensitisation step, as in Example 1, and vice versa.

Electrosensitisation steps and dialysis were carried out as described in Example 1, except that the electrosensitisation steps are carried out twice. Ultrasound sensitivity is determined in a TMM as described in Example 1.

| | | % U/S | |
|---|---|---|---|
| | % U/S | mediated lysis | Mean |
| | mediated lysis | following rest | fluorescence |
| Sample | day of load | overnight | intensity |
| RBC | 0 | 5 | — |
| ES + dialysis | 0 | 0 | 66 |
| (ES + HD) | | | |
| ES + dialysis + | 0 | 90-100% | 61.7 |
| ES | | | |
| (ES + HD + ES) | | | |
| ES + ES + | 84 | 95 | 79 |
| dialysis | | | |
| (ES + ES + HD) | | | |

These results, similar to those obtained in Example 1, indicate that the sequence of the two sensitisations is not critical to obtaining improved sensitivity.

All publications mentioned in the above specification are herein incorporated by reference. Various modifications and variations of the described methods and system of the invention will be apparent to those skilled in the art without departing from the scope and spirit of the invention. Although the invention has been described in connection with specific preferred embodiments, it should be understood that the invention as claimed should not be unduly limited to such specific embodiments. Indeed, various modifications of the described modes for carrying out the invention which are obvious to those skilled in molecular biology or related fields are intended to be within the scope of the following claims.

1. A method for selectively releasing an agent from a red blood cell comprising the steps of:
(a) presensitising said red blood cell;
(b) loading said red blood cell with an agent;
(c) electrosensitising said red blood cell; and
(d) causing said agent to be released from said sensitised red blood cell by applying ultrasound to cause disruption of said sensitised red blood cell, wherein steps (b) and (c) are performed in any order.

2. A method according to claim 1 wherein said electrosensitisation procedures in step (a) and (c) are in vitro or ex-vivo procedures.

3. A method according to claim 1 or claim 2 wherein said electrosensitisation comprises the step of applying an electric field to the red blood cell.

4. A method according to claim 3 wherein said electric field applied to said red blood cells ranges from 0.1 kV/cm to 10 kV/cm under in vitro conditions.

5. A method according to claim 4 wherein said electric field is applied to said red blood cell for 1 μs to 100 ms.

6. A method according to claim 1 wherein said ultrasound is selected from the group consisting of diagnostic ultrasound, therapeutic ultrasound and a combination of diagnostic and therapeutic ultrasound.

7. A method according to claim 6 wherein the applied ultrasound energy source is at a power level that ranges from 0.05 W/cm² to about 100 W/cm².

8. A method for delivering an agent in a vertebrate, comprising the steps of:
(a) presensitising a red blood cell;
(b) loading said red blood cell with an agent;
(c) electrosensitising said red blood cell;
(d) introducing said red blood cell into said vertebrate; and
(e) causing said agent to be released from said sensitised red blood cell by applying ultrasound to cause disruption of said sensitised red blood cell, wherein steps (b) and (c) are performed in any order.

9. A method according to claim 8, wherein said red blood cell is PEGylated prior to being introduced into said vertebrate.

10. A method according to claim 8, wherein said vertebrate is a mammal.

11. A method according to claim 1 or 8 wherein said sensitisation of said red blood cell in step (c) is performed after the loading of said agent in step (b).

12. A method according to claim 1 or 8 wherein said sensitisation of said red blood cell in step (c) is performed before the loading of said agent in step (b).

13. A method according to claim 1 or 8 wherein said loading is performed by osmotic shock.

14. A method according to claim 1 or 8 wherein said agent is selected from a group consisting of a protein, a polypeptide, a peptide, a nucleic acid, a virus, a virus-like particle, a nucleotide, a ribonucleotide, a deoxyribonucleotide, a modified deoxyribonucleotide, a heteroduplex, a nanoparticle, a synthetic analogue of a nucleotide, a synthetic analogue of a ribonucleotide, a modified nucleotide, a modified ribonucleotide, an amino acid, an amino acid analogue, a modified amino acid, a modified amino acid analogue, a steroid, a proteoglycan, a lipid, an oligosaccharide, a glycoprotein and a carbohydrate.

15. A method for preparing a red blood cell composition comprising:
(a) presensitising said red blood cell;
(b) loading said red blood cell with an agent; and
(c) electrosensitising said red blood cell,
wherein steps (b) and (c) are performed in any order.

16. A red blood cell composition obtainable by a method comprising
(a) presensitising said red blood cell;
(b) loading said red blood cell with an agent; and
(c) electrosensitising said red blood cell,
wherein steps (b) and (c) are performed in any order.

17. A kit comprising the red blood cell composition of claim 16, packaging materials therefor and instructions for use.

18. A kit comprising a red blood cell, an agent, packaging materials therefor and instructions for use in a method comprising the steps of:
(a) presensitising said red blood cell;
(b) loading said red blood cell with said agent;
(c) electrosensitising said red blood cell; and
(d) releasing said agent from said sensitised cell by applying ultrasound, wherein steps (b) and (c) are performed in any order.

19. A kit comprising a presensitised red blood cell comprising an agent, packaging materials therefor and instructions for use in a method comprising the steps of:
(a) electrosensitising said red blood cell; and
(b) causing said agent to be released from said sensitised red blood cell by applying ultrasound.

20. A kit comprising a red blood cell composition according to claim 16, packaging materials therefor and instructions for use comprising the step of releasing said agent from said red blood cell by applying ultrasound.

21. The kit of claim 18 or 19, further comprising polyethylene glycol.

22. The kit of claim 16, 18 or 19 further comprising a liquid selected from the group consisting of a buffer, diluent or other excipient.

23. The kit of claim 22, wherein said liquid is selected from the group consisting of a saline buffer, a physiological buffer, serum and plasma.

24. A pharmaceutical composition comprising a red blood cell composition according to claim 16 and a pharmaceutically acceptable carrier or diluent.

25. A device for producing a red blood cell composition of claim 16 comprising:
(a) one or more flow cells and electrosensitisation means; and
(b) one or more dialysis systems;
wherein the flow cell is linked to the dialysis system by connecting means that allows the transfer of red blood cells from the flow cell to the dialysis system and vice versa.
